(19) United States
(12) Patent Application Publication
(10) Pub. No.: US 2025/0180506 A1
(43) Pub. Date: Jun. 5, 2025

(54) Real-Time Contactless Bio-Threat Screening

(71) Applicant: Richard Postrel, Miami Beach, FL (US)

(72) Inventor: Richard Postrel, Miami Beach, FL (US)

(21) Appl. No.: 18/727,101

(22) PCT Filed: Jan. 9, 2023
(86) PCT No.: PCT/US2023/010439

§ 371 Date: Jul. 7, 2024

Related U.S. Application Data
(60) Provisional application No. 63/297,695, filed on Jan. 7, 2022.

(51) Int. Cl. G01N 27/327; G01N 27/30; G01N 33/53

(52) U.S. Cl. CPC G01N 27/3278; G01N 27/308; G01N 27/3276; G01N 33/5308

(57) ABSTRACT

This present invention provides a system, method, and device for rapidly screening individuals at a high rate of speed. The invention features a method, system and device that analyzes an individual's body odors to determine the presence or absence of a disease such as COVID-19 and/or its variants. The invention allows for real-time testing for a pathogen, a disease, or other condition of interest that is especially useful when testing every individual entering or exiting a venue or transitioning through any controlled entry or exit zone demarcated by a portal, passage, security zone, or gate, etc. This testing requires no invasive sampling—or even touch contact—between the device or device operator and the person being tested. The device features a sensing surface whose electronic activity is a function volatile organic compounds (body odor) in the immediate vicinity of its surface.

This invention provides a system, method, and device for rapidly screening individuals at a high rate of speed for infectious disease. The invention features a method, system and device that analyzes an individual's body odors to determine the presence or absence of an infectious disease such as COVID-19 and its variants, hepatitis, measles, tuberculosis, and the like.

The present invention allows for real-time testing for a pathogen, a disease, or other condition of interest that is especially useful when testing large numbers of individuals entering or exiting a venue or transitioning through any controlled entry or exit zone demarcated by a portal, passage, security zone, or gate, etc. This testing requires no invasive sampling—or even touch contact. The device features a sensing surface whose electronic activity is a function of volatile organic compounds (body odor) in the immediate vicinity of its surface. The devices include sensors that are coated to have different specificity characteristics in the manners that they interact with molecules that encounter them. The pattern of interactions detected by the differently coated sensors results in a highly specific multidimensional response pattern for diseases and/or conditions of interest.

Life in living organisms is sustained by the biochemical reactions in their various cells. Depending where a cell is and what its supportive duties are these biochemical reactions will differ. And even the same cell will sport different reactions in response to its changing environments. Thus the biochemical reactions will adjust to the cell's situation at any given time. Life in living organisms is dependent on the biochemical reactions in its living cells with each cell modifying the specific reactions and levels of each in accordance with the conditions in and around each cell. For example, a pathogen will react with particular receptors on particular cells to induce disease specific reactions in the affected cells.

Thus each condition or disease will provoke a unique pattern of cellular responses that result with a disease specific signature of VOCs emanating from the biochemical reactions and emitted through the skin as body odor.

The device features nanosensing elements that are responsive to molecules (VOCs) in their immediate vicinities. At a molecular or atomic scale, the shape of the compound, specifically its electron cloud, determines the manner of interactions with a proximate nanosensing element. The device described for use in this invention comprises a plurality of sensing elements displaying different reactivities (specificities) for VOCs. The sensing surfaces combine a layer of semi-conducting single-walled-carbon nanotubes that obtain differentiated reactivities with VOCs by “decorating” the SWNT surface with compounds to impart selectivity. Different decorating compounds elicit different reaction attributes when coated on a nanosensing element surface. Using a plurality of differently decorated nanosensing elements results in distinguishable compound recognition patterns (signatures) that appear for specific compounds or compound mixtures as gas flows over the sensing elements.

For example, at least two; but preferably a greater number of differently configured surfaces, e.g., 4, 8, 16, 32, 64, 128, 256, 512, 1024, 2048, etc., including all not specifically mentioned intermediate numbers of differently configured surfaces; provide different responses to the same mixture of VOCs exuded from an individual. Each differently configured (decorated) surface is considered a “channel”. The number of available differently configured surfaces (channels) is immense, far greater than the number of sensing elements currently fitting on a chip. A multitude of VOC-sensor surface responses are available.

DNA molecules are a preferred sensor differentiation tool because ssDNA oligomers interact with other organic compounds (such as VOCs) and DNA will durably adhere through I-II interaction to SWNTs in an electrode on a chip surface. The presence of different DNA molecules on a sensing surface differentiates the VOC binding characteristics of that sensor. This allows a sensing specificity to be accomplished by varying the sequence and/or length of the selected ssDNA oligomer molecules on different sensors. Oligonucleotide polymers such as ssDNA oligomer molecules are readily available commercially or from in house synthesis. If an in house source is preferred, oligosynthesizers are available from several vendors, for example, ABI, Beckman, Millipore, Sierra, etc.

ssDNAs, approximately 15 to 100 bases in length, for example, 20 to 30 bases in length, readily and durably attach to the clean SWNT surfaces when a sensor array is prepared. DNAs in this size range are part of a massively large number of different molecules and thus different the population of different binding characteristics is enormous. As an example, if the length of polymers were limited to 24 bases, makes available 4²⁴ or greater than 10¹³ different polymer configurations. Additional differentiation may be achieved by using non-natural bases or different polymer lengths. The number of available differently reacting sensor surfaces is therefore not a limiting factor. To aid the choice of specific ssDNAs, sequence analysis softwares are available for selecting significantly different DNA configurations or avoiding self complementing oligos. Thus, when selecting the polymer sequences for adhering to the SWNTs a designer can take advantage of the extreme variety and choose particular sequences and lengths with different patterns of the bases, and if desired, especially for longer length oligos, can avoid sequences with self-complementing (i.e., folding to form double stranding) tendency.

When the sensors are formed, DNAs in solution are allowed to self adhere to the SWNT stratum. While interaction of the decorated surface with VOCs will be similar with the same DNA adhered to the sensor, different individual sensors with identical DNAs for selectivity will not be molecularly identical. Multiple copies of similarly configured surfaces, e.g., with the same DNA molecules, are thus preferred for reliable data. Similarly decorated sensing elements might be averaged to reduce variability in the data. For example, a predetermined number, e.g., eight, sixteen, or other convenient multiple may be configured as a channel whose members possess identical, or similar attraction attributes, with respect to VOC molecules. Generally, each channel, i.e., each differently configured sensor surface, will have the same number of sensors as other channels. However, if the designer or operator desires, the number of sensors of one configuration channel may differ from another.

The preferred sensor surface is a nanosensing element comprising a surface that electronically interacts with the electronic features of a molecule in sensor's immediate vicinity. In this context “electronic” serves as an adjective form or “electron”. The electrons of a proximal molecule disturb the electronic surface of the nanosensing element. Electron dense zones on VOC molecules will align with electron sparse regions in the DNA decorated surfaces and vice versa. Electrons from the VOCs will repel electrons in the decorations thus altering the electronic characteristics. These altered characteristics are detected in the semi-conducting layer to produce an altered electronic signal. This change is recorded. As discussed above, the altered signals for a single channel may be averaged early in data processing. However, the availability, processing power, cost, and size of data processing units easily permits each sensor signal to be individually collected and processed. The recordings from the nanosensing surface interactions across the multiple channels are collated and analyzed, preferably using machine learning and/or artificial intelligence processes. In this practice, a sampling of emissions from a small number of persons with an identifiable disease or condition (often shortened to “disease” for simplification) serves to train the software associated with the device to recognize a specific disease pattern profile. The disease profile thus formed is tested with each new subject entry and modified with continuing assay results to arrive at a disease “signature” that may be available remotely or incorporated into existing devices with program updates. The VOC recondition pattern from each new patron is compared to the signature(s) of interest to determine if the disease (or condition) is present in that individual.

A large number of the interactions will be with ambient gases and with common human metabolites, not specific to a disease condition. A relatively small fraction of interactions will be relevant to any one screening or detection task. The machine learning processes will help the device ignore the irrelevant or non-contributing interactions and to concentrate on optimizing data collection and processing those interactions illustrative of one or more conditions of interest. In a comprehensive assessment mode, each screening test result is compared to the entire library of profiles and signatures or to entries in a library with signatures for every recognized disease. A test result not matching any library profile or signature may be highlighted as a potential early indication of a novel health threat, such as a zoonotic disease, bio-terrorism, industrial accident, novel disease, virus, or environmental threat. The system may initiate profiling the new disease.

The nanosensing elements are disposed in connection with an interface chip. The elements comprise a surface that interacts with proximal molecules. This interaction changes the electronic properties of the nanosensing surface which alters its relation with the base. In a preferred embodiment the base is at a set voltage, the electromotive force to drive a current. The current is under control of the interaction between the nanosensing surface and a switch such as a field effect transistor or analogous switching circuitry. When the field from the nanosensing element changes, the current flow changes in response. The changes in current flow sensed by each nanosensing element are monitored and recorded for analyzing these interactions.

The time that a VOC lingers in close approximation with the nanosensing surface is one factor for recognizing a type of interaction. The magnitude of change is another. Peak values or values averaged over time of the interaction (with a signal sensed in excess of a predetermined threshold value) are additional factors for consideration in producing disease signatures. In a high discrimination mode, as the VOC approaches the nanosensing surface, a continuous recording of the interactions is analyzed. The VOC's molecular configuration and/or orientation will change as it interacts with and is influenced by the nanosensing surface. The twisting or bending of the VOC can be a feature the device uses to recognize a specific VOC of interest. The high discrimination mode is not an essential feature of the device's functions. Rapid assessment is possible simply assessing slope of magnitude changes or simply change in magnitude over a set time period for each sensor in the several channels with ratios of changes across the channels providing sufficient discrimination to assess several diseases.

The device optimally is configured with a plurality of nanosensing elements in each channel. Not every sensing element will interact with every occurrence of a VOC in a sample. Multiple copies of each type of nanosensing element increases the probability of interactions between a nanosensor surface and a relevant VOC. The redundancy (multiple copies of the same sensor type) increases the sensitivity and selectivity of the device as fewer VOCs pass over the sensing surface without interacting with at least one sensing element. The interaction between the nanosensing surface is transmitted through the sensing element to modulate, e.g., current flow. The bias current and gate current are set in the device and may be identical across all sensing elements, a subpopulation of sensing elements, such as a chip, a row, column, quadrant, or block on a chip, or individual elements on a chip.

The sensing elements are controlled by a controller, preferably a digital to analogue converter that converts computer or algorithmic instructions to produce analogue signals for the each of sensors. As the sensors operate, analogue signals are outputted. An analogue to digital converter accepts the analogue signals and converters each to digital data that a computer analyzes, sorts, and stores. This computer may be on board with one or more sensor arrays, physically attached through an interface with the array outputs, or interfaced with a remote device.

Since the preferred embodiments feature many copies of nanosensing elements, each producing real-time signaling, a streaming device, that coordinates the data into a single data stream for a processor to manipulate, facilitates the data collection, coordination and analysis. Multiple streaming devices may be present in a device. For example, a card with, e.g., 256 nanosensing elements may have its output organized into a single stream or a single stream corresponding to each channel. Multiple cards in a device may each be streamed separately or outputted combined in a single or in parallel streams.

When used in its preferred mode, that is, reading the off-gasses emitted from a preferred body part such as the underside or palm of a hand, the device is used in a real-time analysis setting for high-throughput screening for facility access. The individual's circulatory system delivers nutrients and oxygen to every cell and carries away the cells' metabolites and waste compounds for processing or disposal in the liver kidney, bladder, etc. Gases are delivered from the person into the device and allowed to interact for a predetermined time with the nanosensing element surfaces. These interactions are monitored and screened for relevant interactive data to produce the sample test result (individual profile). The profile is compared with one or more signatures associated with a disease or diseases. Matching comparisons are indicative that the device was exposed to gases associated with that disease or condition, i.e., that the person, whether symptomatic or assymptomatic, likely has that disease.

The device operates when it receives or detects a gas sample from a subject source, e.g., person seeking entry to a venue, for analysis. Nanosensing elements are activated through a digital to analogue converter device that translates machine instructions into device parameters. The gas flow over the elements may be provided by presenting a hand or in other embodiments, another body part, or a sample to the sensor array(s). Simple movement by an individual moving past the device or in some embodiments by a hand passing past, over, or through, or in some embodiments, moving in and out of a marked testing zone can deliver the gas sample for analysis. A fan may augment the gas delivery when faster throughput is warranted.

Such fan may be continuously blowing or it may be activated by a timer, a heat activated switch, a motion activated switch, a light (or diminished light) activated switch, a voice or noise, an operator, etc. The electronic interactions sensed by the sensing elements are recorded and converted to a digital signal that is compiled and streamed to a data processor. The data are processed to arrive as a resulting pattern profile that can be compared to a library of signatures or to a small number of signatures associated with one or more disease of interest. A disease of interest may be associated with a plurality of signatures. For example, an infectious phase and a resolution phase of a disease may be associated with different signatures. Different signatures may be produced by persons presenting with one or more cofactors, for example, male or female, pregnancy, alcohol intoxication, etc. The cofactor may be, but does not need to be identified. Simply, the machine learning algorithms will have associated different signatures with the same disease under different conditions.

A library of VOC profiles and signatures associated with one or more selected diseases or conditions is produced for comparison with the data produced by the device with each sample reading. The profiles and signatures held for comparison in the library of interest are obtained using a learning or teaching process involving a selected group of subjects known to have symptoms associated with the disease or condition of interest and to have a confirmed diagnosis for the disease. Members of this group produce thousands of VOCs, many of which result from healthful biochemical reactions and a fraction of which are particular by their presence, absence, concentration, or ratio with another compound or compounds, in a combination unique to the identified disease. The combination defines the signature for the associated disease. As the device tests additional samples, each definitive result may be used to strengthen the library match process.

In the disease identified sample, many of the VOCs will vary randomly or pseudo randomly. Thus, the disease identified group can act as a control to identify VOCs not associated with the disease and store this information separate from the comparison signature. A group of subjects not identified with a particular disease may have their VOC results used as a negative disease control, i.e., indicative of VOC patterns that are not disease associated. When multiple disease signatures are of interest, the various disease groups can strengthen reliability by serving as negative controls for other diseases.

The preferred embodiment for this invention is its use as a screening device for controlling access to a venue. A person is instructed to place a hand over a screen. When the hand position is acceptable, and sufficient time (e.g., 0.001 to 10 sec, preferably 1 to 5 or 6 sec, more preferably 1-3 sec) the person is instructed or signaled to move the hand away. The simple movement of the hand is often adequate to deliver a sample, from the person to the nanosensors, to produce sample data profiles for comparison to profiles or signatures in the library. The sensing elements often report only normal VOCs (those associated with the human species and not a particular disease or condition) but when signature profiled VOCs are present the device can alert the subject or attendant. The level of normal VOCs detected may serve as an indicator that sufficient subject VOCs have been presented to the device. The device may signal an AllClear™ when a threshold amount of VOCs have been read and no matching disease profile or signature has been observed.

While the device may be set to end the sampling session as soon as the collected data reach a threshold, a set time from beginning to end of sampling session is simpler. When the device detects sample start, either from detecting VOC presence or from a motion detector, object sensor, or the like data collection is set to start. A sampling time of 5 sec is adequate is most applications. A beeper or other signal indicates the testing is complete and results are presented. A test result that does not match a signature of a disease of interest is granted access. Such grant may be indicated by a light, sound or action indicative that the person does not express the disease of concern and accordingly can continue through the access portal to the intended destination. The clearance, e.g., an “AllClear™” notice, may be communicated in any recognizable output signal. The “AllClear™” or lack of positive match signal, may be delivered by any one or several selected means, for example, a green light, a progression of lights along a strip; a positive tone, chime, or RIF; a verbal confirmation or instruction; a gate opening; a gate not closing; a moving walkway; or seat activation; etc. A non-passing result may guide the person to further or secondary testing, deny entry entirely, restrict admittance to an alternate section, etc.

Secondary assessments may be provided in a number of formats. For example, they may require the subject to simply repeat the test a second time or to step into a separate line for a longer time analysis or to use a different hand. The subject may be instructed to remove his or her gloves and present the hand for retest.

Secondary assessments may involve testing gases from another body part. A wand or hand held sniffer connected by an umbilical cord to the device may be used to draw a sample from ambient gas around a portion of the subject and deliver the gas to the device or to a dedicated secondary assessment device. The wand may briefly store a sample for delivery to the device. In some embodiments, a wand may itself include nanosensors similar to those in the primary device, but disposed in a rod or tube allowing sampling from another body area, e.g., the back of the neck, the mouth expelling breath, an armpit, etc.

The device may use the wand as an adjunct sensing tool to be used for screening persons not able to deliver their samples to the sensing module. For example, a person may not have free use of a hand to present to the testing surface. For example, assessing an amputee may require access to a body part other than a hand. An injury may make it impossible, painful, or inconvenient for a subject to present their hand to the device for screening. A toddler or infant may be in a backpack, stroller, carriage, etc., and thus unable to place its hand properly for screening. The wand acts as a tool allowing a device operator or a subject's colleague or guardian to direct the air collecting port in the wand at an alternate body part (or from a hand of a person who is unable to present it themself).

When used, the tip of the wand is directed near the alternate source of the emitted gases. The wand may be in a continuous draw mode as it is guided towards the subject, it may start drawing in gases when moved, it may include a trigger or button to activate gas collection and delivery. The drawn gases continue through the wand to access the sensing chip(s) where screening is effected.

The wand may be used as an adjunct test on non-humans, i.e., to assess object rather than persons. In customs, a disease carried on a parcel, bag, etc., carried by a person would serve as a subject for, e.g., farm borne diseases. An agent would merely point the wand to the area of targeted handbag, etc.

This preferred embodiment would be available for use in a wide variety of placements. It may be set up at any angle, e.g., vertically, horizontally or any angle in between. The nanosensing elements of the device may be configured as a flat surface, flat surface surrounding a recessed area designated for receiving a hand, a curve, a ring, a closed or open shape to accept a hand or other body part-any shape that allows the test subject to deliver emitted off-gasses to the device sensing zones. The device may be designed and delivered for use in many applications including, but not limited to on, in, or connected with: a table or countertop, a turnstile, a wall or barrier defining a walkway, an arched entrance or exit, an escalator, an enclosure (cubical, booth, security scanner, dressing room, outfitting room, phone booth, elevator, etc.), a tent for a meeting or show, a nightclub, a cabaret, a house of worship, a bar, a club, a bank, a cruise ship, a fraternity, a sorority, a funeral parlor, a birthing center, a train, an airplane, a frigate, a cruise ship, counter, a tourist attraction, a theme park, an aircraft carrier, a factory, a subway platform, a public conveyance, a tunnel, a dining room or cafeteria, a restaurant, a hallway, a zone, a secured area, a classroom, a hospital, a courthouse, a coliseum, a stadium, a military installation, an office building, a ferry, a jail, a prison, a voting location, a van, a mobile test site, a mall or retail establishment, a museum, a theater, a cinema, a library, a lobby, a waiting room, customs and immigration checkpoints, a detox center, convention centers, hotels, motels, warehouses, food processing facilities, agribusinesses, space stations, laboratories, etc., in essence any location where people pass through, meet, or congregate. As a person's hand or other body part, e.g., face, breath, etc., becomes proximal to the nanosensing module, gases are driven across the module and analyzed in real-time.

Different venues may incorporate additional features relevant to their missions. For example a concert venue may use the device that includes a ticket scanner, whereby a person seeking entry would require the ticket in addition to e.g., an AllClear™ result for admittance. Other venues may wish to incorporate a physical ID reader, e.g., an RFID, a scanner, a bar code, QR code, palm print, fingerprint, facial recognition, or other personal identification to be checked against databases to confirm that the person has a need to enter at the same time a person receives an AllClear™ designation. A primary application of this invention is for protection and safety of the traveling public which a fundamental component of commerce, both local and international. As such, municipalities, municipal authorities, etc. will benefit from the implementation the technology associated with the present device for use at airports, train stations, bus terminals, ferries, etc. As such, local authorities will use this technology to expedite customer access and movement by combining multiple steps in the security, check-in, validation, etc., in combination with the health screening protocols. Devices that are operated by airlines with access to their Passenger Name Record (PNR) lists to guarantee the safety and security of their passengers and staff may associate travel information with the scanned data. These devices can be used for both departure and arrival, ingress and/or egress. Upon arrival at the destination gate screening may be implemented or repeated to insure local authorities that infected passengers are not arriving in their jurisdictions. When interfaced with the passenger database, the departing passenger could be informed of the terminal or gate of the flight, flight delays-if any, weather at the destination, boarding status, etc. At their destination, the passengers undergoing health screening may be informed of baggage information, connecting flights, messages relating to hotel, rental car, etc. A biometric ID check incorporated with the device may eliminate the need for paper documents, such as ID cards, tickets, boarding passes, baggage checks, etc.

In an office building or hotel lobby, the VOC scan incorporated with personal identification, may instruct the subject to a floor or room number and/or elevator to access the room and ask for a response whether help with baggage or any special treatment is needed. In a warehouse or factory, the punch clock would no longer be necessary. In a waiting room, entry and exit times may be recorded to validate that persons are served in order. A time flag may be built in when a subject fails to exit when expected to either search for that individual or remove them from the queue. In venues when a specific set of individuals is required for a function, the device at the time of access screening can inform other participants of the additional arrival or last person to arrive.

Since assessing the VOCs from the palm is a primary preferred embodiment, the palm print or a portion thereof is a preferred biometric when biometric confirmation is desired. The ID check may interface with other databases, e.g., the Do Not Fly List, or other list of persons seeking entry and alert authorities for possible additional attention. The screening device with its optional adjuncts operates continuously with minimal operator input required. When incorporated with a gating system, the system controls access automatically in response to test results. Personnel requirements to operate the device are low-allowing more efficient assignment for employees. Problems with employee alertness due to shift changes are overcome. Although the access screen primarily concerned with a communicable disease is a primary function of the device, accessory sensors and database access improves access control and customer service while incorporating other tasks involving data obtained during a single scan or reading.

While assessment of metabolic diseases is a primary function of the device, the device may be programmed to operate in a mode where VOCs carried on an individual, an individual's clothing, a package, etc., may be monitored. This program mode looks at content of or recent exposure to possibly dangerous or hazardous molecules including, but not limited to: explosives, explosive ingredients, explosive residue, toxins, environmental markers, etc. In the non-metabolic VOC operations, persons may for example, be screened or cleared to exit an area zone that might have been associated with a release of a toxic substance. In an analogous application, the device may be programmed to screen objects passing by on a belt. The device itself be made motile to pass by or over objects of interest. The movement may be controlled robotically. The wand/umbilical tube embodiment may be used in this manner for example as a tabletop mount, a portal mount, a belt mount, a chest or backpack mount. Controls may be incorporated into the wand itself or in the sensing module of the device. The operator may target the exterior in general, one or more parts of an item, e.g., a seam, an opening a taped area, etc. An open or broken container may be tested to determine degree of hazard to handlers or the public. The programming may be installed in a device to allow switching between detection targets. The device may be configured for a select group of detection targets. Programming for modes not originally installed in the device or accessible to the device may be added using a contact, e.g., plug-in connection, or may be delivered through remote transmission.

While the device is designed for controlling admittance of individuals passing through a checkpoint, the device may be put to use for detecting presence of a condition or disease in a less controlled crowd situation. In a location where a crowd gathers or passes through, the device might not be targeted to screen individual subjects, but perhaps to count the number of passersby presenting with a disease of interest or to a selected group of diseases. This application is particularly useful for public health officials when monitoring the introduction, spread or waning of one or more of the diseases including locations where disease spread is happening or is more likely. One example is a public transport system such as a subway where groups of persons entering and exiting specific stations can be assessed, even anonymously without requiring presenting their hand. Primary sourcing stations and disease stations can be used for mitigating disease spread. Track and Trace is expedited by identifying major sources and locations of likely contact of the infected individuals with others at the locations of disease exit.

Another format may be configured, for example, as a box. A person would be instructed to place a hand in or near the box whereupon a sampling of gases emitted from the hand is fed to the vicinity of the nanosensing elements. The hand would remain until the person was instructed to proceed. The instruction may be an ALLCLEAR™ instruction for admittance to the venue or alternative instruction for secondary or further actions or assessments to address concerns relating to the disease raising the alarm.

However formatted, the device is or is part of a system that comprises: a control component, a sensing component, a data compiling and delivery component, an analytical component, an information storage component, and a reporting component. The primary sensors are preferably present in multi-channel arrays of nanosensing elements that monitor VOCs. Other sensors may include operational features including, but not limited to: an on/off switch, a WiFi or Bluetooth detector, a motion detector, a temperature sensor, a proximity sensor, a time sensor, a global positioning sensor, a light detector, a sound detector, etc. The primary sensors react to the electronic properties of a molecule in close proximity. Weak influences, attractive or repulsive result in a change in the electronic characteristics on the sensor surface. Different VOCs will influence and be influenced by the nanosensing surfaces. These changes or interactions will be repeatable for the same molecules closely approximating the sensor surface.

In a preferred configuration, multiple copies of similarly “decorated” sensors will be activated in an analysis. The decorations or functionalizing compounds interact with the VOCs. Aromatic, cyclic hydrocarbons and heterocyclic compounds frequently interact with a large variety of VOCs. The heterocyclic rings in the nucleic acids, preferred as functionalizing compounds, assist in securing the nucleic acid to conductive base. Carbon containing organic heterocyclic components of the nucleic acids are available to interact with VOCs which activate sensor elements to produce a signal. Nucleic acids are additionally a preferred decoration because of their variability and ease of production. Different sequences of the four natural nucleotides, for example in a sequence with just eight nucleotides, provide over 50,000 available sequence structures to choose from. As the number of bases in the functionalizing increases. Using synthetic nucleotides in addition to the four, increases the number of choices available. Decorations may also be fabricated with different lengths further magnifying the number available for selection.

Even though many sequences can interact with a given VOC, different decorations provide for differential interactions and thereby are tools for optimizing differentiation capacity, selectivity and sensitivity. Merely incorporating different decorations on different sensing elements in the device provides differential identification between two or more similar VOCs. In a refined embodiment, specific VOCs of interest are identified and decorations, including those that may not be nucleic acids, are selected to more selectively interact with those VOCs.

A suitable conductive base comprises elemental carbon. Single layer planes or tubes of carbon are especially advantageous for their retention of decorations such as biologic molecules, which contain a lot of carbon atoms, but also for compactness and capacity to transmit electronic fluctuations occurring as decorations are affected by proximal electronic (VOC) influences. Single layer two dimensional graphene may also be used in some embodiments by themselves or in conjunction with Single Wall carbon NanoTubes (SWNTs). Crumpling the graphene so that it does not lay flat can increase surface area and allow for 3-dimensional fitting of decoration molecules. SWNTs with a tubular carbon structure that can lay flat on a surface like graphene or be made in a three dimensional format when SWNTs may have one end resting on the base with another end elevated on top of one or more SWNTs are a preferred carbon base for supporting decorations.

The nanosensing elements can be of a variety of preselected sizes (diameters), heights (due to stacking density), shapes and configurations. Generally a nanosensing element is decorated with a single species of biomolecule, often a nucleic acid such as RNA or DNA with a length between 8 and 35 nucleotides, more preferably between 20 and 30 in length. RNA is another available decoration. The single layered carbon supports generally accept cyclic, especially polycyclic compounds, e.g., porphyrins, phthalocyanines, and azobenzene, as non-covalent associations on their surfaces. Polymers such as the nucleic acids, polyethylene glycol, and fatty acids-especially those conjugated with short polypeptides also form stable surface interactions with the single layered carbon structures and thus are available choices as functional groups on the nanosensing element surfaces. Modified nucleic acids with e.g., nucleotides other than the standard A, C, G, and T and/or incorporation of other molecules in the complex, e.g., stable nucleic acid lipid particles (SNALPS), offer additional choices for detection variations. The assembly of SWNTs decorated with such addons is well-known in the art. Therefore one skilled in the art does not require a repeated teaching in this paper.

The different channels may be differentiated by microdeposition of different nucleic acid molecules preselected for each channel. Such DNA polymer molecules may be adhered to the SWNT substrate by exposing a nonosensor pad to a droplet containing DNA in aqueous solution.

Different DNA molecules are easily manufactured according to selected sequences. For example, a length of 24 bases using one of the 4 natural bases available for each position allows for >a million-million different strands. If desired non-natural bases are available to provide even more differentiation of sensor sensitivity. Nucleic acid polymers will readily form non-covalent bonds with the carbon nanotubes providing connectivity between the electrodes on each individual sensor. The different sequences will curve in accordance with their base sequences (chemical structure) and bond to the SWNTs using π-π stacking to present a variety of 3 dimensional shapes atop the sensor surface.

The sensors are preferably set on arrays. Each array is supported on a chip such as the commonly used Si wafer. Photolithographic patterning is an adequate process for patterning the chips. A lift-off process with 5 nm Cr/40 nm is acceptable for forming a back gate. Etching is an alternative process if desired. The Atomic Layer Deposition process benefits from Physical Vapor Deposition of aluminum.

A hafnium oxide layer can be deposited using atomic layer deposition. A number of cycles is necessary to achieve a desired ˜85 nm thick layer. Several hundred may be necessary, with the actual number of cycles determined by the thickness of the growing layer.

The array is then patterned with photolithographic etching Photoresist spin-coating with polymethylglutarimide (PMGI) @ 3000 rpm, 45 s, then a baking @ 180° C., 5 min, followed with S1813 @ 4000 rpm, 45 s, and baking @ 115° C., 1 min with an exposure dose of 70 mJ/cm². The plate is then developed in AZ-300 MIF, for 45 s. The patterning continues with reactive ion etching (RIE) using CHF₃ 40 sccm+Ar 20 sccm about 20 minutes depending on the Hf) 2 thickness. Descumming follows using an O₂ plasma (20 sccm) for 1 min.

The residue from photoeresist treatment can be removed at this time to maintain HfO₂ availability for the carbon nanotube deposition. Soaking the plates in n-methyl-2-pyrrolidone (NMP) for a (long overnight, ˜15 hours) followed by an IPA (2-propanol) rinse and a water rinse. Two additional NMP soak/rinses are performed at 60° C. with each NMP rinse followed with an IPA then a water rinse. Annealing can be accomplished using 1000 sccm Ar+250 sccm H₂ at 220° C. for ˜60 min. N₂ carrying 5% H₂ can serve as an alternative.

Electrodes can be formed using photolithography. A 5 nm Cr/40 nm Au lift of process is performed prior to a second NMP rinse process to clear the photoresist residue. The bond pads can be formed using another photoresist stage with 100 nm Au followed by a lift-off and a third residue removal NMP rinse. Carbon nanotubes (>99.9% semiconducting) (obtained from the manufacturer in tolune) are then deposited in the toluene for coating the array sensor surfaces. The misdeposited SWNTs can be removed using photoresist patterning. O₂ plasma (90 sccm, 1 min, 60 W) is sufficient to remove the unwanted SWNT deposits. A final annealing similar to the prior annealing prepares the plates for dicing out the arrays.

The sensors on the arrays are then treated with a selected specificity agent. DNA polymers because of their ease of manufacture, stability, affinity for VOC gases, and propensity for n-bonding to the nanotubes is a coating of choice. Selected ssDNAs can be prepared in an approximately 100 nM solution and distributed as single droplets onto each individual sensor pad. 30-60 min allows the DNA oligomers to distribute across and bind to the nanotube surfaces. A sensor array with 16 channels of 16 sensors per channel thus would be treated using 256 droplets appropriately positioned. Drying must be accomplished without flashing droplet fragments across to a neighbor sensing pad.

These methods are presented as adequate teachings for making the electrode arrays. The technology sector has developed improved high definition-high throughput manufacturing procedures. Such improved technology and further expected improvements are not necessary to form the sensor arrays, but if desired, could be applied.

The device may be configured as a circuit card or a collection of circuit cards. Each card supports a plurality of sensing elements both physically and interconnectively. A digital to analogue converter may power the nanosensing operations. Digital instructions are converted to dictate or influence analogue values, for example of temperature, bias voltage, gate voltage, etc., on the nanosensing elements. For example, an array or array support may receive a digital instruction to maintain an analogue temperature approximating 24° C. A temperature sensor may report to the initial temperature control instructor to apply heating or cooling influences to the array. An analogue output fan may be an effector under the digital control instructor.

An analogue to digital converter accepts input from the nanosensing elements and produces digitized data. The data are organized and streamed by a controller of the outputted signals. Inputs from a plurality of simultaneous measurements, e.g., current fluctuations, are organized and combined in a stream of data for computer analysis. Interfaces that connect the different functions and components are provided.

A plurality of computers may be interfaced to work in or with the system. For example a NUC computer may process the sensor results in an on board operation. The on board computer may interface hard wired or remotely with one or more adjunct computers, e.g., at a testing station or remotely in a central collection and processing facility (e.g., for data security) or across outside computational support such as a “cloud” service vendor.

Power can be through a battery or remote delivery, that is provided by a source outside the main device cabinet. Power delivered through an external source is preferably delivered through a device battery to remove electrical noise. The outside power will often be delivered electrically through conductive cabling, but other means of power transmission, e.g., inductive pickup, light beam, solar panel, etc., can work as well. A gate voltage serves as differential that allows current to flow. The current flows, in response to alterations in a bias voltage that changes in response to electronic interactions between the VOC and the nanosensor and the nanosensor support. These voltages maybe under directions delivered through the digital to analogue converter.

A bias voltage may be selected as optimized depending on the nanosensing elements and supports chosen. A 100 mV bias is compatible with many devices. But the allowable range may span several orders of magnitude. For example bias voltages in the order of 10 mV, 1 mV, 100 μV, 10 μV, 1 μV, or even in the nV range may find some applications. Similarly, bias voltages greater than 100 mV, e.g., in the order of 1 V, 10 V, 100 V, 1,000 V may be useful in other applications.

A gate voltage provides the differential to feed the current. A value of −3 V is easy to achieve and corresponds to two regular alkaline batteries. As capacitance is reduced, e.g., by reducing insulating layer thickness, the gate voltage may be reduced. The current flow is a design choice meaning that the direction of flow (changing voltage form plus to minus) depends on configuration choices. A voltage in the order 1 V, 0.3 V, 5 V, 9 V, 12 V, 18 V, 20 V, 24 V, 30 V may be selected.

An exemplary nanosensing cartridge may comprise 256 nanosensing elements arranged in a 16×16 grid. While each element or a selection fraction of elements, e.g., a 4×4, or 8×8 block, a column or a row, may be individually manufactured to have its unique decoration, the ease of manufacture and advantages of redundancy suggest building chips of: e.g., 256, 400, 512, 1024, etc., with the same decoration molecule or complex. Individual elements or groups of elements may present with different temperature or bias voltages though on the singly decorated chip so not all elements will interact identically.

A circuit card holding a chip cartridge with multiple, e.g., 16 sensing blocks, can serve as a component central to the sensing operations. For example, 256 element chips my be arranged in a 4×4, 4×8, 4×16, 2×4, 2×8, 2×16, or other selected geometry. In some embodiments chips may be disposed on opposite sides of a circuit card. The number of elements on a chip is a design choice. This example using 256 was chosen for its analogy to computer circuitry where powers of 2 are common. Continuing with this theme designers may prefer chips with 128, 512, 1024, 2048, 4096, etc., sensing elements. Incorporating sensing elements on both sides of a chip, e.g., top and bottom, is an obvious method for doubling. When chips are square, a factor of 4 would be observed. However, the designer is not bound to follow these conventions, a chip with any convenient number of sensors is possible, e.g., 25, 81, 625, etc., 15, 18, 21, 24, 27, 30, 33, 36, 39, 42, 51, 54, 57, 60, etc., 36, 49, 64, 100, etc., etc. Designers may choose any convenient arrangement and number of chips on a card or in a device. For example, square arrangements of chips, 3×3, 4×4, 5×5, 6×6, 7×7, 8×8, 9×9, etc., may be convenient. Columns of chips, e.g., 2, 3, 4, 5, 6, 7, 8, etc., may be chosen for a rectangular arrangement. The rows likewise can be of a number convenient to the designer. Powers of 2 are common for most technological operations.

In the configuration described above, 4 cards may serve as box walls, with opposing sides having sensor components facing one another. Since 1, 2, 3, or 4 sensor cards are sufficient with additional cards adding to redundancy, this style of disposition allows for the card or cards with the greatest signal (perhaps closest to the hand or closer to the tissue with best off-gassing) to signal data collection endpoints when a threshold of VOCs has been detected. Pentagons, rectangles, hexagons, triangles, curved shapes, regular or irregular are suitable designs for housing the sensing components. Additional redundancy improves durability (time between maintenance) and has a valuable feature of allowing cross checking during calibration cycles. The number of sensors and separate sensor chips is therefore a design choice. If a single chip or even a single card appears faulty, the redundancy allows the device to continue operations. Redundancy is a design choice to be decided by the designer, manufacturer, purchaser, regulative body, etc.

The sensing circuit cards are not confined to box shaped disposition. The sensing plate or card may be situated on or recessed in a surface vertically or horizontally, e.g., in a countertop or railing as an example for assessing persons on a walkway or moving walkway. A zone adjacent to a doorbell or an elevator button may be outfitted with a sensing module. The device may be portable, i.e., available for a person to carry in a case. The device may be fixed in a building structure or as part of an entryway to a building or part thereof. A portable device may include attachments allowing vertical, horizontal or other angle disposition. Any flat or curved surface may be configured as a device retainer and therefore a screening location. When multiple sensor components are present, some may be arranged in a parallel orientation for simultaneous subject exposure, others may be arranged in series such that opportunity for exposure of the subject gases to the sensor components is along a time sequence.

The device envisioned is programmable in one or more respects. A simple programming operation involves activating or turning on the device. The device is also controllable through the electronics and permitted access of gases to the sensors. The sensors or data collection, analytical and/or storage functions may be under program control, for example, by operator interface, subject interface, motion, detection, time interval, a screening sensor that activates other sensing components or functions, a remote control, etc. The program may be selected from a collection of programs stored for the machine to select from. Programs may be delivered from a centralized location such as a company, home office, a local office, a security, room, an inserted instructional or datacard, etc. The interface carrying the program may feature a component including, but not limited to: a keyboard, a touchscreen, a chip (such as a USB chip) a microwave transmitter and receiver, a WiFi connection, Bluetooth, an e-pen, a port for plugging in a chip, a phone, connection, a computer, a computer memory and program storage device, an app on a connected or remote electronic device, a dedicated tap and touch pad or screen, a microphone with voice deciphering capability, biometric security modules (e.g., face recognition, voice recognition, fingerprint, keyboard patterns, etc.). Additional security may include features including, but not limited to: access coding from a person or machine, encryption, confirmation required by a system manager, etc.

The programming may be as simple as activating the device or a portion thereof. The programming may select one or more diseases for matching. The programming may incorporate additional learning cycles. The programming may interactively adjust one or more sensor's parameters such as adjusting temperature, a voltage, a weighting applied to a sensor's or a group of sensor's outputs, a screen that may divert gas or direct the gas more strongly at a particular sensor or group of sensors.

The device is adaptable and programmable for application in different environments or applications. Outdoors or in environments with flow, a windscreen may be affixed on the device to facilitate capture of subject emitted gases by shielding the zone surrounding the sensing area from convection currents that may convey the VOCs past or away from the sensing elements. Lighting may help direct the subject to the test device and/or direct the subject how and where to place or move the hand. Light intensity may be increased for dimly lit access areas or for outdoor use at night.

Disease libraries with their profiles and/or signatures can be added or deleted as concern for a disease grows or wanes. Program updates may be frequent for some diseases as variants change prevalence. Emerging diseases, such as COVID-19 in the early 2020s, can be incorporated in the programming as the disease becomes recognized. Different venues may feature different programming. For example, a positive test at some venues may simply advise the subject and deny entry. In a different venue, an escort may be summoned to guide the subject for further assessment or treatment.

The programming in a particular device may direct comparison to a single disease or condition of interest, to a select group of diseases, to diseases with associated signatures, to a set of toxins, or other comparisons selected by the user. Program updates to add or adjust profiles or signatures can keep the device current. Programming may be effected using any acceptable interface or interface method. Programming may require a hardwired or plug in device when strict control is observed. Wireless updates may be accomplished automatically or on request.

While the methods related to using the device do not require identification of the molecular structure of the VOCs or other compounds detected, knowledge of specific molecules that are involved in a disease signature can act as a factor in improving the device. Identifying the compounds instrumental in forming the disease signatures can be applied to e.g., selecting a decoration or decorations for the sensing elements, adding scavenger compounds to differentiate between similar compounds, adjusting a voltage or temperature, adding a pre-read filter, etc. For example, time for evaluating each subject sample, sensitivity and/or selectivity of the device, may be refined by chemical analysis, including, but not limited to: x-ray fluorescence spectrometry, ion chromatography, gas chromatography, gas chromatography-mass spectrometry, inductively coupled plasma mass spectrometry, inductively coupled plasma optical emission spectroscopy, scanning electron microscopy, x-ray diffraction, electron probe microanalysis, nuclear magnetic resonance, etc.

The device may be part of a system of devices where the experiences of each device are delivered into a cloud or other inclusive data set. The system continues to analyze the data and notifies device operators when updates are available or when instructed by the operator or in concert with regulations delivers downloads to the relevant devices.

Additional assessments may involve a screen that does not involve VOCs as tested by the device, including, but not limited to: a biosample (e.g., PCR or antigen), a thermal reading, an x-ray, etc.

### EXAMPLE

A 16 channel chip is formed with 16 sensors in each channel. In this hypothetical example, QC determines that channel 2, element 7 produces flatline data; channel 5, elements 2 and 15 produce a significantly noisy signal; channel 12, element 5 produces a signal that fails to correlate with other channel 12 element signals. In this example, although the data from every sample are collected, during subsequent analysis these outliers are ignored. In this example, the data analysis system may recognize outliers up to 25% of the sensors in a channel in the default mode and continues to operate with 12 of the 16 sensors in any channel within tolerance. An alarm-message to a monitor, indicator lamp in or on a device, remote signal to a maintenance depot, a beep, or other interface, is preferably initiated when a channel drops below 90% of properly functioning sensors.

During an operational session, e.g., over the period of hours, days, or weeks, the sensors baselines may drift. For example, during a daily operation, a signal, e.g., current flow, may see its initiation point decline over multiple readings. To address such drift, each measurement initiation point is processed as a “zero” with changed signal, e.g., reduced current, recorded as a percentage or ratio of the initiation current. In a 16 channel system with 16 sensors per channel as an example, a single processor chip can scan through each sensor in a channel, and subsequent channels in series. In this example, a single processing chip may read a sensor over, e.g., about a 300-500 usec sampling time, move to the next sensor to accomplish a channel assessment in about 100 msec. In this example, using 400 usec time to sample each sensor, each sensor is scanned about every 0.1 seconds. In this example, over a 5-6 sec time period used to read and analyze the gases from the individual's hand, each thus each sensor is scanned and processed >50 times. The electronics in the device preferably use off-the-shelf data processing chips. Currently available chips that are available at low cost are easily capable of achieving reading and analysis rates greater than 2⁸×10² which produces 100+ data points per second from each sensor. While the ˜400 usec pace for reading each sensor can provide adequate data for the device to deliver accurate output, higher rate scans, e.g., >˜2.5×10³ sensors read per second may improve device performance, especially with respect to auto-diagnostics.

Sensor scanning preferably continues after the individual is instructed to remove their hand from the sampling zone to monitor the device's return to an initiation state.

Although not considered an optimal embodiment, signal from a number of sensors in a channel, e.g., groups of 2, 4, 8, 16, or the entire channel may be averaged using either digital or analogue data collection for signal processing. Although such system may be simpler to construct and maintain, aberrant sensor monitoring would require a separate standardization step. Current off-the-shelf chips easily independently assess each sensor output.

As an example, a sensing device with a palm sized air permeable covered zone is configured with at least one array of sensor chips, e.g., 16 channels of 16 sensors each beneath the port zone. Multiple arrays may be installed in a device. Optionally, a second, third, or fourth array remains covered until selected for analysis. For example, if a first array has performed outside acceptable limits. In such example a second array may be opened when, e.g., channel 6 of a first array is functioning with only 13 sensors. Dual sensing arrays may continue operation with the data processing downgrading the below standard channel(s), e.g., array 1, channel 6, In a multi arrayed device all arrays may function providing the unit with redundancy. As any sensor or channel deviates or drifts below acceptable standards, the redundant channels will be adequate to maintain sensing tasks.

A n exemplary two array system may operate normally with both arrays in normal operation. But if an array or one or more channels in any array become compromised, the redundancy allows compromised outputs to be ignored while retaining adequate sensing performance.

The box functions when a person waves their had across the sensing port gases emitted through the skin on the hand are drawn into the sensing device. One or more fans may facilitate delivery of the VOCs from the hand. One or more fans may focus the gases onto the array(s). A fan may exhaust gases from the unit, drawing the intake from the zone with the waving hand, through the sensing port, and across the array(s). While diffusion can be adequate for feeding the sensors, using a fan can decrease analysis time and increase throughput.

In one embodiment a motion or object detector senses approach of a hand. The data storage and analysis system is turned on. Preferable the system remains active; it is just the data storage and analysis that is activated. In the quiescent state, minimal data processing may continue to continuously self-assess the sensors's integrity. Optionally one or more fans are turned on or increased in their draw. The approach sensor may be of any available format, e.g., active or passive infrared, acoustic or ultrasonic, a motion detecting camera, etc.

### Sensing Example

A person approaches the scanning station and is instructed verbally, graphically, textually, etc., to place the hand in the sensing zone, e.g., above the box with the sensing port. A motion or proximity sensor such as an active infrared diode system, detects the approach and position of the hand as it prepares for and initiates signal collection and processing. A preferred distance between the hand and the port is in a range about 30 to 50 mm. The signal collection may be entirely electronic or may incorporate a physical collection add-on such as a fan. In some embodiments, the device may incorporate an infrared system for monitoring hand temperature. These data may be inputted into the analysis algorithm, e.g., during comparison of ratios from the outputs from different channels. Some embodiments may incorporate a radiant heater to slight warm the hand to increase volatilization of the VOC gases. Radiant heat may be transmitted through infrared LEDs or other sources. In extreme conditions, such as in wintry or arctic environments, the device may pause while the hand is heated for a predetermined time or to a predetermined temperature. The person is instructed to maintain the hand in sensing position over the sensing port. A timer is activated when the hand is in position. After the predetermined sensing time, e.g., 5 sec, 6 sec, 7.5 sec, 8 sec, 10 sec, or another time chosen by the device operator, a signal instructs the person to remove their hand from the sensing station and to proceed as instructed. The system continues to flush or refresh awaiting the next sensing action.

When the device signals absence of a condition (such as infectious disease) that the system has been programmed for, the person may be instructed to proceed along their intended course. However, if a hazard such as an infectious disease is apparent the person will be instructed in another direction. In addition to pathologic agents the device might be programmed to screen for presence of or exposure to toxins or non-pathologic disease such as cancers or auto-immune disease.

After the device has processed data and signaled results, the flow rate is optionally increased, e.g., by accelerating a fan or engaging an additional fan. This interim period serves to flush the sensors in preparation for the next analysis. The system may be programmed to return to a background or quiescent state after a predetermined time period.

The different channels are differentiated by microdeposition of different nucleic acid molecules for each channel. Such DNA polymer molecules may be adhered to the SWNT substrate in aqueous solution. DNA ˜100 nM is pipetted sprayed printed or otherwise deposited on the a layer of carbon nanotubes and allowed to interact 30-60 minutes e.g., ˜45 minutes before allowing to dry.

The selected different ssDNA molecules are easily manufactured and prepared for delivery to the nanosensing surfaces in accordance with the predetermined sequences. A number largely in excess of the number of available channels serves as potential decorations on the nanosensing surfaces. For example, a length of 24 bases using one of the 4 natural bases available for each position allows for >a million-million different strands. If desired non-natural bases are available to provide even more differentiation of sensor sensitivity. Nucleic acid polymers will readily form non-covalent bonds with the carbon nanotubes providing connectivity between the electrodes on each individual sensor. The different sequences will curve in accordance with their base sequences (chemical structure) and bond to the SWNTs using π-π stacking to present a variety of 3 dimensional shapes atop the sensor surface.

The sensors in their channels are preferably set on arrays. Each array is supported on a chip, such as the commonly used Si wafer. Photolithographic patterning is an accepted process for patterning the chips. A lift-off process with 5 nm Cr/40 nm is acceptable for forming a back gate. Etching is an alternative process if desired. The Atomic Layer Deposition process benefits from Physical Vapor Deposition of aluminum.

A hafnium oxide layer can be deposited using atomic layer deposition. A number of cycles is necessary to achieve a desired ˜85 nm thick layer. Several hundred may be necessary, with the actual number of cycles determined by the thickness of the growing layer.

The array is then patterned with lithographic etching, photoresist spin-coating with polymethylglutarimide (PMGI) @ 3000 rpm, 45 s, then a baking @ 180° C., 5 min, followed with S1813 @ 4000 rpm, 45 s, and baking @ 115° C., 1 min with an exposure dose of 70 mJ/cm². The plate is then developed in AZ-300 MIF, for 45 s. The patterning continues e.g., with wet chemical etching, vapor etching, or reactive ion etching (RIE) using CHF₃ 40 sccm+Ar 20 sccm about 20 minutes depending on the Hf), thickness. Descumming follows using an O₂ plasma (20 sccm) for 1 min.

The residue from photoeresist treatment can be removed at this time to maintain HfO₂ availability for the carbon nanotube deposition. Soaking the plates in n-methyl-2-pyrrolidone (NMP) for a (long overnight, ˜15 hours) followed by an IPA (2-propanol) rinse and a water rinse. Two additional NMP soak/rinses are performed at 60° C. with each NMP rinse followed with an IPA then a water rinse. Annealing can be accomplished using 1000 sccm Ar+250 sccm H₂ at 220° C. for ˜60 min. N₂ carrying 5% H₂ can serve as an alternative.

Electrodes can be formed using lithography, e.g., common photolithography. A 5 nm Cr/40 nm Au lift of process is performed prior to a second NMP rinse process to clear the photoresist residue. The bond pads can be formed using another photoresist stage with 100 nm Au or a CMOS compatible metal followed by a lift-off and a third residue removal NMP rinse. Carbon nanotubes (>99.9% semiconducting) (obtained from the manufacturer in tolune or other SWNT solvent) are then deposited in the toluene for coating the array sensor surfaces. The misdeposited SWNTs can be removed using photoresist patterning. O₂ plasma (90 sccm, 1 min, 60 W) is sufficient to remove the unwanted SWNT deposits. A final annealing similar to the prior annealing prepares the plates for dicing out the arrays.

The sensors on the arrays are then treated with a selected specificity agent. DNA polymers because of their ease of manufacture, stability, affinity for VOC gases, and propensity for n-bonding to the nanotubes is a coating of choice. Selected DNAs can be prepared in an approximately 100 nM solution and distributed as single droplets onto each individual sensor pad. 30-60 min allows the DNAs to distribute across and bind to the nanotube surfaces. A sensor array with 16 channels of 16 sensors per channel thus would be treated using 256 droplets appropriately positioned. Drying must be accomplished without flashing droplet fragments across to a neighbor sensing pad of a different channel. Aerodynamic force, e.g., accomplished with a jet of compressed gas or a vacuum may remove the unwanted liquid and remnant (not adhered) oligos.

These methods are presented as adequate teachings for making the electrode arrays. The technology sector has developed improved high definition-high throughput manufacturing procedures. Such improved technology and further expected improvements are not necessary to form the sensor arrays, but if desired, could be applied.

1. A device capable of real-time, contactless screening for disease or other biohazard, said device comprising:
a container housing at least one array of nanosensing surfaces;
said container comprising a port permeable to ambient gases including body odors emitted from an individual;
said at least one array supporting a plurality of nanosensing surfaces; said nanosensing surfaces comprise a layer of single-walled carbon nanotubes (SWNTs) on an electrode substrate;
said plurality of nanosensing surfaces comprising distinct channels having different affinities for volatile organic compounds (VOCs);
sad distinct channels having different affinities comprising different oligonucleotide molecules, wherein each of said distinct channels comprises a single set of identical oligonucleotide molecules adhered to that channel's SWNT layers;
said nanosensing surfaces in electronic contact with a data collector, compiler, and analyzer;
at least one power system providing power enabling communication through said electronic contact and providing power to said data collector, compiler, and analyzer;
said analyzer comprising at least one algorithm capable of communication with a data repository and comparing data resulting from an analytical event with data in said data repository; and
at least one indicator capable of signaling completion of said analysis event.

2. The device of claim 1 wherein said different oligonucleotides comprise different DNAs.

3. The device of claim 2 wherein said DNAs are selected from oligonucleotides having between 20 and 30 nucleotide bases.

4. The device of claim 3 wherein at least two channels have 24 nucleotide bases.

5. The device of claim 1 further comprising a fan that draws a flow of ambient gas through said port to said array.

6. The device of claim 1 further comprising a detector that detects approach or presence of a hand.

7. The device of claim 6 wherein said detector operates using light or sound.

8. The device of claim 7 wherein said detector operated using passive or active infrared light.

9. The device of claim 6 wherein said detector switches at least one activity in said device.

10. The device of claim 9 wherein said activity is selected from the group consisting of: a timer, a fan, a data processor, and a light.

11. The device of claim 10 wherein said light comprises an infrared radiant heater.

12. The device of claim 6 wherein said detected presence is less than or equal to about 50 mm from said port.

13. The device of claim 6 wherein said detector detecting said presence comprises signal from at least one of said nanosensing surfaces.

14. The device of claim 1 comprising at least 16 distinct channels.

15. The device of claim 1 wherein a channel comprises at least 16 nanosensing surfaces.

16. The device of claim 1 wherein said port has an area between ˜16 and ˜225 cm².

17. The device of claim 10 wherein said port has an area 100 cm² plus or minus ˜10 cm².

18. The device of claim 1 wherein said container houses two arrays of nanosensing surfaces.

19. The device of claim 1 wherein a fan is associated with each array.

20. A method for real-time, contactless screening for disease or other biohazard, said method comprising:
monitoring VOC emissions emitted from an individual's skin by collecting electronic information from a nanosensing device;
said information responsive to said monitored emissions;
processing said electronic information to form an electronic profile corresponding to said electron monitored VOC emissions;
comparing said profile to at least one VOC content signature associated with a disease;
and indicating presence or absence of a match of said profile with said signature.

21. The method of claim 20 wherein said monitoring occurs over a period of time in a range between ˜0.001 sec and ˜10 sec.

22. The method of claim 21 wherein said range is between ˜4 sec and ˜6 sec.

23. The method of claim 22 wherein said range approximates 5 sec.

24. The method of claim 21 wherein a time between monitoring VOC emissions from a first individual and monitoring VOC emissions from a second individual is greater than or equal to about 2 times the range of time for said monitoring.

25. The method of claim 21 wherein a minimum time between monitoring VOC emissions from a first individual and monitoring VOC emissions from a second individual is between 10 and 30 sec.

26. A method of preparing an array of VOC nanosensors on a chip, said method comprising:
patterning a substrate wafer to form gates and gate dielectric films;
depositing a metallic layer on said patterned wafer to form source and drain electrodes;
selectively removing dielectric films to form electrical contacts;
lithographically patterning electrodes atop said metallic layer;
lithographically patterning bond pad regions;
coating said wafer with SWNTs;
lithographically patterning said coating of SWNTs;
functionalizing said coating of SWNTs comprising: delivering a solution of selected ssDNA oligomer molecules to the SWNTs that are atop the nanosensor undergoing fabrication, allowing said selected ssDNA oligomer to partition to the SWNT layer, and removing said solution; and
during preparation of said array, singulating and wire bonding said wafer to form an array chip.

27. The method of claim 26 wherein said substrate comprises oxidized silicon.

28. The method of claim 26 wherein said metallic layer depositing comprises depositing hafnium oxide.

29. The method of claim 26 wherein said selectively removing comprises at least one process selected from the group consisting of: reactive ion etching, wet chemical etching, and vapor etching.

30. The method of claim 26 wherein said photolithographically patterning bond pad regions comprises depositing Au or at least one CMOS compatible metal.

31. The method of claim 26 wherein said coating with SWNTs comprising immersing said wafer or a part thereof in a solution comprising SWNTs in toluene or other solvent for said SWNTs.

32. The method of claim 20 wherein at least one of said processing and comparing comprises machine learning or artificial intelligence.

33. The method of claim 26 wherein a time of partitioning is between about 30 and about 60 minutes.

34. The method of claim 26 wherein said removing is accomplished with a jet of compressed gas.
